(19) United States
(12) Patent Application Publication
(10) Pub. No.: US 2022/0362574 A1
(43) Pub. Date: Nov. 17, 2022

(54) DEVICE AND METHOD FOR TREATING CONDITIONS USING ELECTROMAGNETIC RADIATION

(71) Applicant: FLUO LABS LLC, Camden, DE (US)

(72) Inventors: Jan Enemaerke, Camden, DE (US); Lawrence Johnson, Camden, DE (US)

(73) Assignee: FLUO LABS LLC, Camden, DE (US)

(21) Appl. No.: 17/868,714

(22) Filed: Jul. 19, 2022

Related U.S. Application Data
(63) Continuation of application No. PCT/US22/11505, filed on Jan. 6, 2022.
(60) Provisional application No. 63/134,583, filed on Jan. 6, 2021.

(51) Int. Cl. A61N 5/067

(52) U.S. Cl. CPC A61N 5/067; A61N 2005/0663

(57) ABSTRACT

A device and method for therapeutically treating conditions using electromagnetic radiation (EMR) is described herein. EMR, of optimal wavelengths and at surprisingly low energy levels, are pulsed for a time duration when aimed at an area of interest, the outcome being an improvement in the condition while not doing damage to surrounding tissue.

### CROSS-REFERENCE TO RELATED APPLICATIONS

This application claims priority from U.S. Provisional Application No. 63/134,583, filed on Jan. 6, 2021, and PCT Patent Application PCT/US22/11505, filed on Jan. 6, 2022, all of which are incorporated by reference herein in their entirety for all purposes.

### STATEMENT REGARDING FEDERALLY SPONSORED RESEARCH OR DEVELOPMENT

Not Applicable.

### FIELD

The present teachings relate to a device and methods that use electromagnetic radiation for therapeutic purposes.

### INTRODUCTION

The power of electromagnetic radiation (EMR) healing has been the subject of intense interest in the last 20 years thanks to scientific discoveries that now provide an understanding of the mechanism through which EMR, especially infrared and red light, enhances cell metabolism through the process of photobiomodulation. The therapeutic use of electromagnetic radiation is mainly known for treating skin conditions, but electromagnetic radiation may also be used to treat other medical conditions, such as respiratory conditions.

While older products provide the use of electromagnetic radiation for treating conditions such as allergic rhinitis, the energy levels used in related devices deliver energy on the order of Joules per centimeter squared, use broad spectrum light, and deliver heat which may lead to unintended tissue damage. The greater the energy level delivered by such devices, the greater the risk for damaging tissue, including thermal damage.

For example, the Allergy Reliever SN206 (Lloyds Pharmacy Ltd., Coventry, U.K.) emits infrared light delivering 0.54 Joules/cm² per three-minute cycle. The manufacturer claims that the 652 nm and 940 nm infrared light delivered via the nasal probes suppresses release of histamine promotes and increased blood flow respectively

BIONASE™ is a patented medical device manufactured by Syro Technologies Ltd. (Jaffa, Israel). This device emits a visible red light at a single wavelength of 660±5 nm. The manufacturer's website (www.biolight.co.il) recommended starting treatment three times daily and then titrated according to symptomatic response. Each treatment session lasts for 4.5 minutes and the device switches off automatically. To date, only one study has been published evaluating the efficacy of BIONASE™ in treating perennial allergic rhinitis and nasal polyposis. A study using this device states that subjects received “ . . . intranasal illumination at 660 nm for 4.4 minutes three times a day for 14 days (total dose 6 Joules per day). A push-button switch on the control box activates the probes for 4.4 minutes, during which time 1 Joule of light energy is delivered from each unit. Patients were instructed to introduce the probes into their nostrils as deeply as possible and to press the push button. Each nostril was subjected to low-energy stimulation (4 mW) for 4.4 minutes (1 Joule per treatment session) three times a day for 14 consecutive days).

Conversely, other light therapy devices in the market are based on design considerations using an older generation of light, optics and battery technology which invariably translates in a poor consumer experience. Many are low-powered requiring several minutes of administration to achieve modest results. Those operating at a single wavelength miss out on some of the potential benefits of photobiomodulation that multiple wavelengths can provide. Finally, any device using UV or blue light present serious safety issues. None of the other older devices are cleared for marketing in the U.S. by the Food and Drug Administration.

Developing a device and treatment for multiple therapeutic uses that is quickly administered and does not higher energy levels would be of great benefit.

### SUMMARY

A device is provided for treatment of a medical condition, the device comprising a body, an electromagnetic radiation source within the body that generates electromagnetic radiation of at least two discreet, non-contiguous wavelengths from about 400 nm to about 1000 nm, an applicator, optionally at least one skin contact sensor adjacent to the applicator, and a power source providing power to the electromagnetic radiation source. The applicator may be at least one of a delivery nozzle and a wand, with the delivery nozzle optionally taking on a conical shape and the wand optionally taking on a cylindrical shape. The converter may comprise a number of components, namely a printed circuit board, an integrated circuit, at least one resistor, a linear voltage regulator, a dual-in-line switch, an audio jack, a power barrel connector jack, at least one LED light, and a connector receptacle.

The electromagnetic radiation wavelengths include at least one wavelength from about 400-700 nm, about 850-870 nm, and about 920-950 nm, and in various embodiments the electromagnetic radiation wavelengths are about 660 nm, about 850 nm, and about 940 nm. In various embodiments, the electromagnetic radiation source optionally emits a broad spectrum white light in addition to the wavelengths above. In yet other embodiments, the electromagnetic radiation source is at least one of a laser and a light-emitting diode.

The device can further comprise an on/off button, a cap, and a controller board. The controller board can comprise at least one of a triggering unit, a capacitor, and electronic timing circuitry.

In various embodiments, the applicator is interchangeable with a second applicator that emits a different electromagnetic radiation wavelength or combination of electromagnetic radiation wavelengths. The electromagnetic radiation source can emit the electromagnetic radiation wavelengths based on programming from the controller board, with the electromagnetic radiation wavelengths being selectable.

The electromagnetic radiation can be emitted as a pulse between about 1 pulse per treatment and about 30 pulses per treatment. The pulse duration can range from about 1 millisecond to about 1000 milliseconds, and in various embodiments the pulse duration ranges from about 10 milliseconds to about 100 milliseconds. The frequency of pulses ranges between about 1 Hertz and about 10 Hertz.

The electromagnetic radiation may be applied from about 0.5 mJ/cm² to about 0.5 J/cm², and in various embodiments the electromagnetic radiation may be applied from about 0.75 mJ/cm² to about 0.25 J/cm², and in particular the electromagnetic radiation may be applied at about 1.0 mJ/cm². In various embodiments, the electromagnetic radiation source is applied within 60 mm from a distal end of the applicator.

A method of treating a condition is also provided using the device described herein. The method includes:
i. providing a device for treatment of a condition, the device comprising:
ii. aiming the applicator towards an area of interest; and
iii. emitting electromagnetic radiation from the applicator at the at least one wavelength from about 400 nm to about 1000 nm.

The electromagnetic radiation is emitted as at least one pulse, with the at least one pulse duration ranging from about 1 millisecond to about 1000 milliseconds, and in various embodiments the at least one pulse duration ranges from about 10 milliseconds to about 100 milliseconds.

The electromagnetic radiation can be specified so that its wavelength falls within a predefined range. In various embodiments, the electromagnetic radiation is non-thermal. The electromagnetic radiation may be applied from about 0.5 mJ/cm² to about 0.5 J/cm², and in various embodiments the electromagnetic radiation may be applied from about 0.75 mJ/cm² to about 0.25 J/cm² and in yet other embodiments about 1.0 mJ/cm².

The medical condition treated by the method may be at least one of allergic rhinitis, non-allergic rhinitis, perennial allergies, seasonal allergies, ragweed allergies, sinusitis, cold sores, acne vulgaris, and herpes. A kit is also provided comprising the device described herein and instructions that explain how to use the device.

In one example, a device for treatment of a medical condition of a user is provided comprising a body, a delivery nozzle, at least one light emitting diode in the delivery nozzle that generates electromagnetic radiation of at least two discreet, non-contiguous wavelengths including from about 660 nm, about 850 nm, and about 940 nm, wherein the electromagnetic radiation is emitted as a pulse between about 1 pulse per treatment and about 60 pulses per treatment, and wherein frequency of pulses ranges between about 1 Hertz and about 10 Hertz, and optionally at least one skin contact sensor adjacent to the applicator; and a power source providing power to the electromagnetic radiation source, wherein the electromagnetic radiation is be applied to a treatment site of the user from about 0.5 mJ/cm² to about 0.5 J/cm².

### DRAWINGS

Those of skill in the art will understand that the drawings, described below, are for illustrative purposes only. The drawings are not intended to limit the scope of the present teachings in any way.

FIG. 1. A side cross-sectional view of the device.

FIG. 2. A perspective view of the device.

FIG. 3. A back view of the device.

FIG. 4. A side view of the device.

FIG. 5. A front view of the device showing the on-off button.

FIG. 6. A front view of the device with the cap removed.

FIG. 7. A front cross-sectional view of the device.

FIG. 8. A graph of PBMCs pre-treatment and post-treatment with a proxy of the present device.

### DETAILED DESCRIPTION

### Abbreviations and Definitions

To facilitate understanding of the invention, a number of terms and abbreviations as used herein are defined below as follows:

Treatment: As used herein, the term “treatment” or “treating” may include alleviating, minimizing, managing, ameliorating, suppressing, eradicating, reducing the severity of, decreasing the frequency of incidence of, preventing, reducing the risk of, and/or delaying the onset of a disease or condition. In various embodiments, the treatment may be targeted to the underlying disease or condition and not to condition symptoms or to ancillary pathologic processes that are not directly related to the underlying condition. In various other embodiments, the treatment may target the underlying condition, condition symptoms, and ancillary pathological processes or any combination thereof.

User: As used herein, the term “user” refers to the person who is administering the EMR from the device, or an animal to which the device is being administered.

Device and Methods for Treating Conditions Using Electromagnetic Radiation

Unlike older devices that may cumulatively administer 5, 10, 15, and even more than 20 Joules of light over several minutes to several days, the present invention provides a therapeutic treatment with much less energy and much less time. The present device delivers therapeutic dosages of electromagnetic radiation in periods of milliseconds per pulse instead of continuously over seconds or minutes. The present device delivers a therapeutic dosage, but in shorter time. In particular, this allows the treatment to be provided with less energy, including cumulative energy over the course of a multi-day treatment. An advantage of using lower energy for treatment of certain medical conditions is that the present device is well below the thermal threshold, beyond which tissue damage can occur.

Without being bound to a particular theory, it is believed that cells absorb EMR at certain wavelengths to positively benefit those with certain medical conditions, one of which is allergic rhinitis. Mitochondria specifically are believed to be involved with the improvement of symptoms caused by these medical conditions. Even more specifically, cytochrome c oxidase, found in mitochondria, is a photoacceptor, able to absorb EMR. EMR absorption by mitochondria has been found to stimulate increased proton electrochemical potential, ATP synthesis, increased RNA and protein synthesis and increases in oxygen consumption, mitochondrial membrane potential, and enhanced synthesis of NADH and ATP, all of which may have a positive effect on alleviating symptoms from certain conditions. In addition, EMR absorption by cells is believed to reduce the antigen-presenting capacity of cells and inhibits synthesis and release of pro-inflammatory mediators from several cell types. Indeed, it has been shown that pro-inflammatory mediators such as histamine have been decreased when human inflammatory cells are exposed to EMR at certain wavelengths. With EMR exposure, there is also an increase in IL-10, a natural dampener of inflammation.

Many older products intended to deliver light therapy produce thermal effects which may unintentionally harm tissue which is not intended for therapy. In part, this is because the older products use higher energy light sources that create thermal effects. These are unintended side effects which older products cannot reduce.

Unlike older products, the present device provides therapy at an energy lever less than half of what was previously thought possible. That is very surprising and also teaches away from certain theories in which higher light energies are needed to produce a therapeutic effect. The present invention uses much lower energy over a shorter amount of time.

The present invention provides a device for the treatment of a user's medical condition comprising: a body, an electromagnetic radiation (EMR) source that generates at least one pulse of electromagnetic radiation of at least one wavelength from about 400 nm to about 1000 nm, an applicator, and a power source providing power to the EMR source. Optionally, at least one skin contact sensor or other sensor that can detect the distance from the device and a site of therapy can be present on or adjacent to the applicator. Such optional sensor is not depicted in the figures herein.

In accordance with an aspect of the present teachings, the EMR is provided by the device to deliver a dosage per pulse from about 0.5 mJ/cm² to about 0.5 J/cm² to the user's site of treatment. More specifically, the dosage per pulse of the EMR may range from about 1 mJ/cm² to about 0.4 J/cm², from about 1.2 mJ/cm² to about 0.3 J/cm², from about 1.3 mJ/cm² to about 0.2 J/cm², from about 1.4 mJ/cm² to about 0.1 J/cm², and from about 0.75 mJ/cm² to about 0.25 J/cm². In various examples, the dosage per pulse of the EMR may be about 117 mJ/cm², about 7.4 mJ/cm², 1.4 mJ/cm², or 1.0 mJ/cm². The total amount of energy delivered to a site of therapy is much lower older products over a certain amount of time.

In accordance with yet another aspect, the electromagnetic radiation is non-thermal. The electromagnetic radiation does not damage tissue once impacting the tissue. For example, the EMR delivered to an area of interest does not exceed about 45 degrees Celsius. In another example, the EMR delivered to an area of interest ranges between about 10 degrees Celsius and about 45 degrees Celsius, between about 15 degrees Celsius and about 45 degrees Celsius, between about 20 degrees Celsius and about 45 degrees Celsius, between about 25 degrees Celsius and about 45 degrees Celsius, between about 30 degrees Celsius and about 45 degrees Celsius, between about 35 degrees Celsius and about 45 degrees Celsius, and between about 40 degrees Celsius and about 45 degrees Celsius.

In accordance with another aspect, the device further comprises a controller board. The controller board is housed within the body of the device. For example, the controller board can be programmed to emit EMR from the EMR source. The controller board can comprise at least one of a triggering unit, a capacitor, and electronic timing circuitry. The function of the electronic timing circuitry is timing treatment frequency and/or dosage.

In accordance with a further aspect, the electromagnetic radiation wavelengths are emitted from the EMR source based on programming from the controller board, with the EMR wavelengths being selectable. For example, the controller board may be programmed to deliver certain predetermined wavelengths, duration, dosage, and pulse structure. In another example, a user may select which wavelengths are emitted from the EMR source. In yet another example, the EMR source is capable of delivering two or more discrete, non-contiguous EMR wavelengths.

With respect to the electromagnetic radiation range of wavelengths of the EMR source, the range of wavelengths is between about 480 nm and 1000 nm. For example, the electromagnetic radiation range of the EMR source can range between about 610 nm and about 980 nm, between about 620 nm and about 960 nm, and between about 630 nm and about 940 nm.

In accordance with a further aspect, when the EMR source emits EMR in multiple discrete, non-contiguous ranges, the electromagnetic radiation wavelengths can include a wavelength from about 480-700 nm, another from about 850-870 nm, and another from about 920-950 nm. In another example, the EMR wavelengths can include a wavelength from about 500-700 nm, from about 850-870 nm, and from about 935-950 nm. In yet another example, the EMR wavelengths include at least one wavelength from about 600-700 nm, from about 850-870 nm, and from about 940-950 nm. In addition, the EMR wavelengths can include at least one wavelength from about 650-700 nm, from about 850-870 nm, and from about 940-950 nm. The EMR can deliver all wavelengths simultaneously, or in a series.

More specifically, and in accordance with a further aspect, the EMR source emits EMR in three discreet ranges from about 655 nm to about 665 nm, from about 845 nm to about 855 nm, and from about 935 nm to about 945 nm. In another example, the EMR source emits EMR from about 656 nm to about 664 nm, from about 846 nm to about 854 nm, and from about 936 nm to about 944 nm. In another example, the EMR source emits EMR from about 657 nm to about 663 nm, from about 847 nm to about 853 nm, and from about 937 nm to about 943 nm. In another example, the EMR source emits EMR from about 658 nm to about 662 nm, from about 848 nm to about 852 nm, and from about 938 nm to about 942 nm. In another example, the EMR source emits EMR from about 659 to about 661 nm, from about 849 nm to about 851 nm, and from about 939 nm to about 941 nm. In yet another example, the EMR source emits three wavelengths including about 660 nm, about 850 nm, and about 940 nm. Those of skill in the art understand that such discreet wavelengths can be provided, for example, by light emitting diodes and lasers. Any EMR capable of providing such discreet wavelengths is intended to be within the scope of the present invention.

It is known that, generally, near infrared light (˜750 nm) is extinguished more than 5 mm beneath the surface of the tissue or skin, red light (˜650 nm) is extinguished some 4-5 mm beneath the surface of the tissue or skin, yellow light (˜585 nm) about 4 mm, and green light (˜540 nm) between 2-3 mm, blue light (˜440 nm) barely 1 mm into tissue, whereas ultraviolet light (˜350 nm) hardly penetrates at all. The reason such discreet wavelengths extinguish at different depths is that skin consists of a range of chromophores which have scattering and absorption coefficients which are highly wavelength dependent. The scattering properties of tissue are due to attenuation properties intrinsic to the chromophore and also to the size of the particles within the tissue which also governs the type of scattering that occurs, namely Mie or Rayleigh scattering. Scattering leads to light dispersion in the tissue and the eventual reduction in the energy density with increasing depth.

White light (having a contiguous, broad spectrum wavelength pattern comprising wavelengths from 400-700 nm) has a low depth of penetration and is not therapeutic according to the teachings of the present invention. Combinations of discreet wavelengths (e.g., 660 nm+/−5 nm), on the other hand, have been discovered to be therapeutic in conjunction with the device of the present invention. Different medical conditions may be treated using different combinations of discreet wavelengths being applied to sites of therapy. For example, allergic rhinitis may be treated using a first combination of wavelengths, and a viral infection may be treated using a second combination. The combinations may be selected based on the skin or tissue depth of penetration desired for treatment.

In accordance with yet a further aspect, the device optionally has an electromagnetic radiation source that emits a broad spectrum white light in addition to the electromagnetic radiation wavelengths that includes at least one wavelength from about 480-700 nm, about 850-870 nm, and about 920-950 nm. The purpose of this optional non-therapeutic white light source is to illuminate or provide a guide for inserting or applying the applicator to a site of therapy.

In accordance with a further aspect, the electromagnetic radiation source is one of a laser, a light-emitting diode (LED), or a combination thereof. All pertinent wavelength ranges listed above may be generated from the electromagnetic radiation source. One of skill in the art would recognize that any EMR source that is capable of generating one or more discrete wavelengths between about 600 nm and about 1000 nm would be suitable for the device, which may include delivery by fiber-optical communication to a position near the user's site of therapy.

In accordance with yet another aspect, the electromagnetic radiation is emitted as a pulse between about 1 pulse per treatment and about 30 pulses per treatment, depending on the medical condition to be treated. A typical treatment may be between 1 millisecond to several minutes.

More specifically, the EMR is delivered between about 3 pulses and about 60 pulses per treatment, between about 12 pulses and about 30 pulses per treatment, between about 14 pulses and about 28 pulses per treatment, between about 15 pulses and about 27 pulses per treatment, between about 16 pulses and about 26 pulses per treatment, between about 17 pulses and about 25 pulses per treatment, and between about 18 pulses and about 24 pulses per treatment. One of skill in the art would recognize that different medical conditions would require potentially different treatment regimens using the device, and that such regimens can be determined by a medical practitioner using ordinary skill. The treatment can be repeated over a number of days depending on the regimen.

In accordance with yet another aspect, the pulse duration ranges from about 1 millisecond to about 1000 milliseconds. For example, the pulse duration may range from about 1 millisecond to about 900 milliseconds, from about 10 milliseconds to about 800 milliseconds, from about 20 milliseconds to about 700 milliseconds, from about 30 milliseconds to about 600 milliseconds, from about 40 milliseconds to about 500 milliseconds, from about 50 milliseconds to about 400 milliseconds, from about 60 milliseconds to about 300 milliseconds, and from about 70 milliseconds to about 200 milliseconds. In various aspects, the pulse duration need not be delivered using a consistent pattern and may change, for example, from 60 milliseconds to 300 milliseconds and then to 150 milliseconds.

In accordance with yet another aspect, the electromagnetic radiation is emitted as a series of pulses, with individual pulse durations ranging from about 10 milliseconds to about 100 milliseconds. More specifically, the pulse duration may range from about 9 milliseconds to about 90 milliseconds, from about 8 milliseconds to about 80 milliseconds, from about 7 milliseconds to about 70 milliseconds, from about 6 milliseconds to about 60 milliseconds, from about 5 milliseconds to about 50 milliseconds, from about 4 milliseconds to about 40 milliseconds, and from about 3 milliseconds to about 30 milliseconds. In various aspects, the pulse duration need not be delivered using a consistent pattern and may change, for example, from 10 milliseconds to 100 milliseconds and then to 50 milliseconds.

In accordance with yet a further aspect, the frequency of pulses ranges between about 1 Hertz and about 10 Hertz. More specifically, the frequency of pulses ranges between about 2 Hertz and about 9 Hertz, between about 3 Hertz and about 8 Hertz, and between about 4 Hertz and about 7 Hertz. In various aspects, the pulses need not be delivered using a consistent pattern and may change, for example, from 3 Hz to 8 Hz and to 5 Hz.

In accordance with yet another aspect, the electromagnetic radiation is delivered to the user's site of therapy within about 60 mm from a distal end of the applicator. More specifically, the electromagnetic radiation is delivered to the user's site of therapy within between about 0 mm and about 50 mm from a distal end of the applicator, between about 0 mm and about 40 mm from a distal end of the applicator, between about 0 mm and about 30 mm from a distal end of the applicator, between about 0 mm and about 20 mm from a distal end of the applicator, and between about 0 mm and about 10 mm from a distal end of the applicator. In various examples, the electromagnetic radiation is delivered from about 0 mm from a distal end of the applicator, about 25 mm from a distal end of the applicator, and about 50 mm from a distal end of the applicator. In various aspects, the distances above may change, for example, from a distance of 60 mm for a first treatment, to 10 mm for a second treatment, and 20 mm for a third treatment, and so on. A skin contact sensor, or other sensor providing information to the user as to the distance the applicator is from the site of therapy, may optionally be combined with the applicator to assist the user in determining the optimal or predetermined distance between the site of therapy and the applicator. The sensor may be, for example, a flange on the applicator which contacts the site of therapy at a predetermined distance, or an electronic sensor which is triggered at a predetermined distance from the site of therapy.

In accordance with yet another aspect, a light reflector resides within the applicator.

In accordance with yet another aspect, the applicator is interchangeable with another applicator. For example, there may be multiple applicators, each able to deliver different EMR wavelengths or sets of EMR wavelengths. The applicators may be used interchangeably depending on the condition being treated; in other words, one applicator may be for a first condition (e.g., to treat allergies), and another applicator may be for a second condition (e.g., to treat a viral infection). In another aspect, the substitution of the applicators may change variables that affect the treatment regimen, such as wavelength, pulse dosage, treatment duration, and power. The substitution of the applicators may change the emitted wavelength or set of wavelengths. In another example, the substitution of the applicators may change pulse dosage. In another example, the substitution of the applicators may change treatment duration. In another example, the substitution of the applicators may change power to the EMR source.

In yet another aspect, each applicator may be designed for a particular number of treatments and disposed of when all such treatments have been consumed by a user. In another example, the number of treatments may be set based on frequency of use, severity of symptoms, and for a set period, after which the applicator would be disposed of. In yet another example, the number of treatments may be set based on frequency of use, after which the applicator would be disposed of. In another example, the number of treatments may be set based on severity of symptoms, after which the applicator would be disposed of. In yet another example, the number of treatments may be set for a set period, after which the applicator would be disposed of.

In another aspect, the body of the device may be held in a user's hand, and preferably within the user's fingers. In another aspect, the applicator and power source can reside at opposite ends of the device, or at the same end of the device. In another example, the electromagnetic source is housed within the body. The power source status indicator indicates the power source status, and as an example, the indicator shows a different color to indicate the device being on but with low battery, the power source is charging, and the device is fully charged. In another example, the indicator shows a flashing light when the device is on but with low battery, and solid light when the device is charging. In yet another example, the indicator shows different solid colors when the device is charging and the device is fully charged. The indicator may flash a color when the device is on but the battery is low, may be a solid color when the power source is charging, and may be another solid color when the power source is fully charged.

In accordance with yet another aspect, the device comprises a cap. For example, the cap covers the tip of the applicator and may be disposed of after usage. In another example, the cap is attached to the body, resting on the body when the device is not in use. One of skill in the art would recognize that the cap may attach to the body in myriad ways. For example, the cap snaps onto the body when the device is not in use.

In accordance with a further aspect, the applicator may take the form of at least one of a delivery nozzle and a wand. The delivery nozzle is optionally a conical shape or cylindrical shape. One of ordinary skill in the art would recognize that the shape of the delivery nozzle and wand could take on multiple forms, just as long as the form does not interfere with the delivery of EMR to an area of interest.

In accordance with yet another aspect, the device further comprises an on/off switch or button. The function of the switch or button is starting and stopping electromagnetic radiation emission. The switch or button is on the exterior surface of the device. The device may be set to be rendered unusable from between about 2 seconds and about 60 seconds after use. More specifically, the device may be set to be rendered unusable from between about 5 seconds and about 60 seconds, about 10 seconds and about 60 seconds, about 15 seconds and about 60 seconds, about 30 seconds and about 60 seconds, or about 45 seconds and about 60 seconds after use.

In yet another aspect, once the device is ready to deliver EMR, an audible signal ranging from about 50 dB to about 90 dB is emitted at least once. More specifically, the audible signal range can be from about 55 dB to about 90 dB, from about 60 dB to about 90 dB, from about 65 dB to about 90 dB, from about 70 dB to about 90 dB, from about 75 dB to about 90 dB, from about 80 dB to about 90 dB, and from about 85 dB to about 90 dB. Pressing the on/off button at this point starts the EMR emission and treatment. In another example, pressing the button twice within one second will turn the device off. In another example, holding down the on/off button will turn the device off. In yet another example, the device will turn off if left unattended, and in particular if left unattended from about 30 seconds to about one minute. In yet another example, the audible signal can alert a user that the treatment session in one nostril is finished, and they can move the device to the other nostril.

The present teachings also include a method of treating a user's medical condition using the device above. The user aims the device applicator towards an area of interest, for example, a site of therapy. The area of interest can be the user nostril for treatment of a number of respiratory conditions including allergies and viral infections. In various examples, the area of interest is an ear cavity, the mouth, a specific area of user skin, a user sinus cavity, a mucous membrane, or various orifices found on the user body.

The device is then used to emit electromagnetic radiation from the applicator at one or more wavelengths that range between about 400 nm to about 1000 nm to impact the area of interest using one or more pulses, each pulse providing a dosage per pulse from about 0.5 mJ/cm² to about 0.5 J/cm² to the user's site of treatment. While not being bound by a particular theory, the electromagnetic radiation in certain wavelengths typically initiates photobiomodulation to take place, as described herein. It is thought that photobiomodulation generates a photochemical effect whereby electromagnetic radiation is absorbed by cells in the body to create chemical change. The transformation of light to energy triggers a complex set of physical and chemical reactions that enhances the cells' performance and responses. The precise balance of electromagnetic radiation wavelength, dosage, power, and pulse structure effectively impedes phenomenon like the allergic cascade.

In accordance with yet another aspect, the electromagnetic radiation is provided within a predefined range. In another example, the device employs filters to block certain wavelengths from emitting.

In accordance with yet another aspect, the condition that the device treats may be at least one of allergic rhinitis, non-allergic rhinitis, seasonal allergies, perennial allergies, ragweed allergies, sinusitis, cold sores, acne vulgaris, and herpes. One of skill in the art would recognize that other conditions may be treated with the device so long as the conditions respond favorably to EMR wavelengths ranging from about 400 nm to about 1000 nm.

The present teachings also include a kit for treating a condition, the kit comprising the device described above for treatment of a medical condition described above and instructions that explain how to use the device.

These and other features, aspects and advantages of the present teachings will become better understood with reference to the following description, examples and appended claims.

Hand-Held Device Form Factor

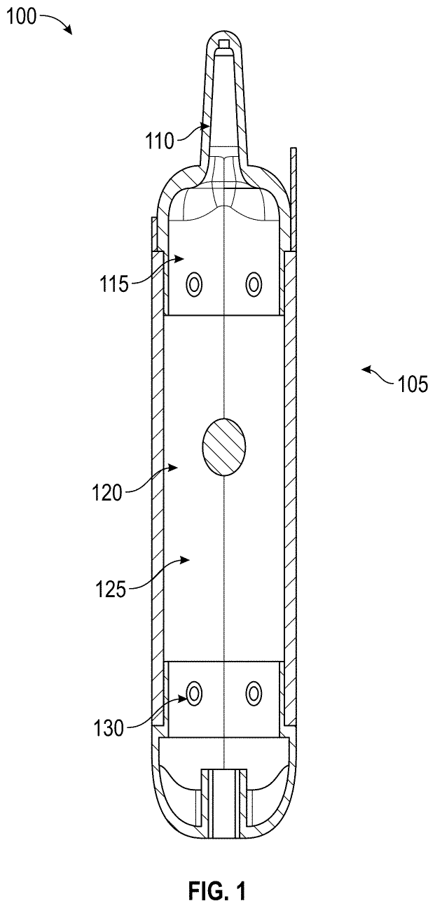

The present invention is directed to a device 100 comprising a body 105, which contains the electromagnetic radiation source 115, a controller board 125, and a power source 130, as seen in FIG. 1. One of skill in the art will recognize that this form factor is one of a number of form factors that can be used. The device may also be made of any material suitable to be manufactured in a hand-held form. An on/off button 120 is on the exterior surface of the body 105. An applicator 110 connects to the body 105. In an embodiment, the applicator 110 is contiguous with the body 105 and is not removable. In another embodiment, the applicator 110 may be removed from the body 105 and is interchangeable with other applicators.

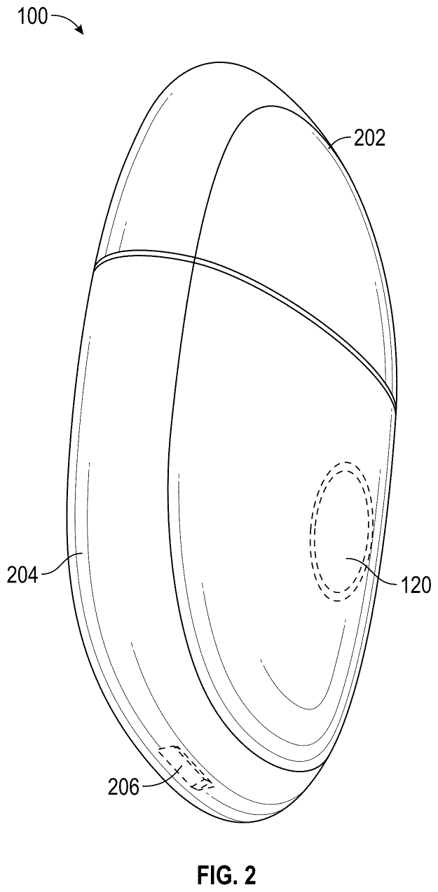

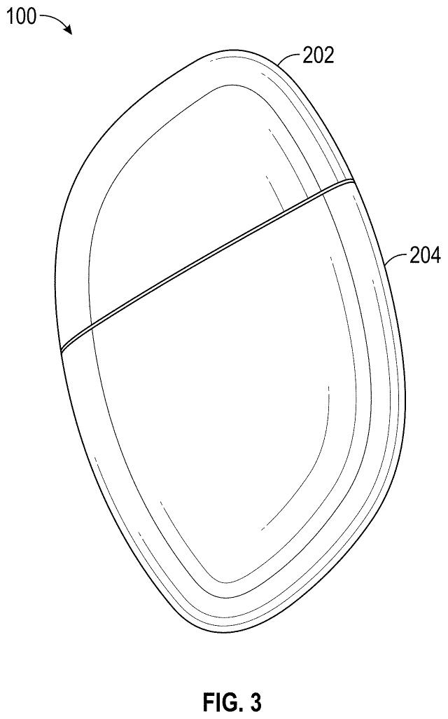

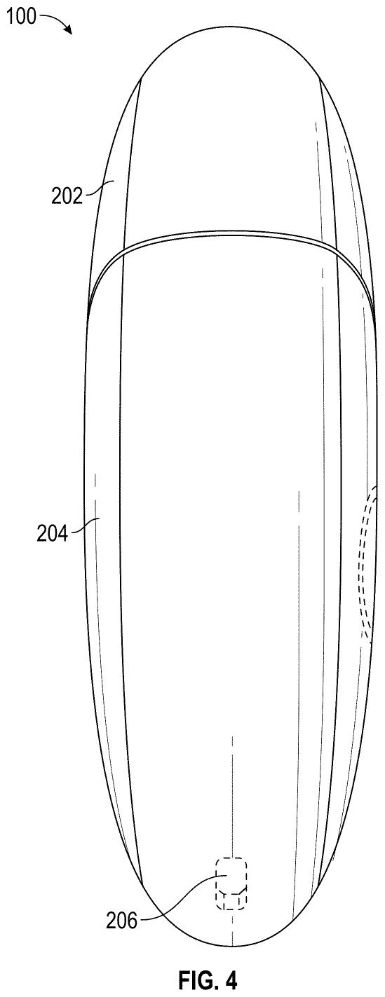

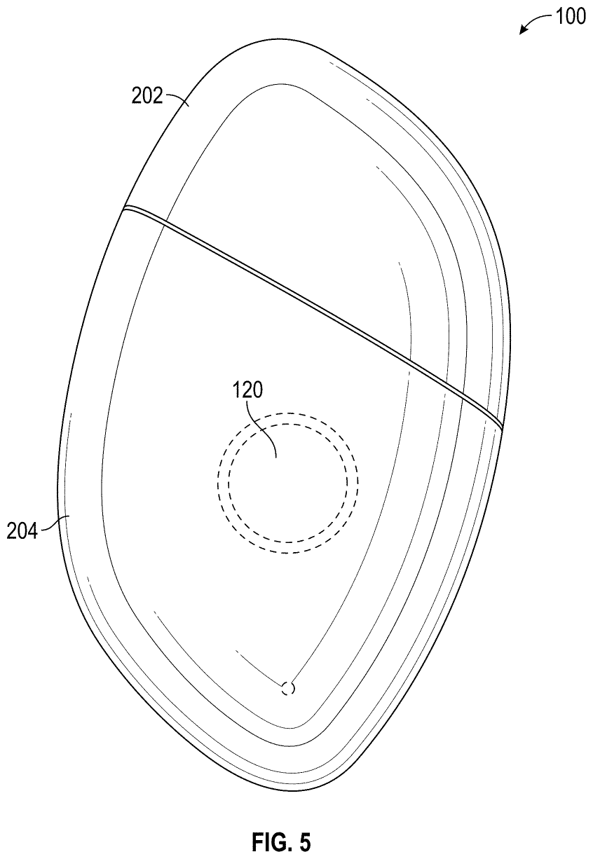

FIGS. 2, 3, 4 and 5 show various views of the device 100. The device 100 has a body 204 and a cap 202, which is removable. A charging outlet 206 that allows the device 100 to be charged so that it functions may be near the base of the body 204. In a non-limiting example, the charging outlet 206 may be near the upper region of the body 204. In a non-limiting example, the charging outlet 206 may be on the side surface of the body 204. In a non-limiting example, the charging outlet 206 may be on a face of the body 204. The on-off switch 120 is on a face of the device, positioned near the middle of the face. In a non-limiting example, the on-off switch 120 is in the upper region of a face of the device. In a non-limiting example, the on-off switch 120 is in the lower regions of a face of the device.

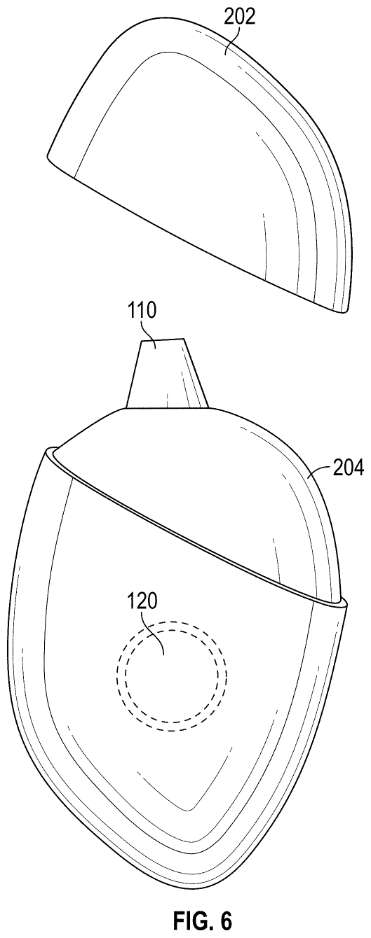

FIG. 6 shows a front view of the device 100 with the cap 202 removed from the body 204. Once the cap 202 is removed, the applicator 110 may be placed in position for EMR to be delivered to the area of interest. In a non-limiting example, the device 100 has no cap 202. In a non-limiting example, there is a cap 202 that fits onto the body 204 when not in use.

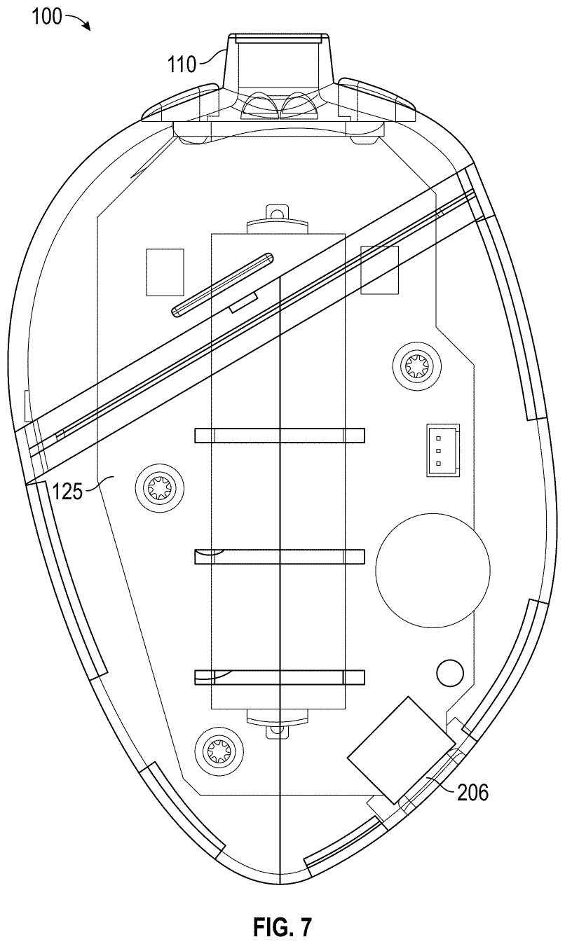

FIG. 7 shows a front cross-sectional view of the device 100, showing the applicator 110, controller board 125, and charging outlet 206. The controller board 125 is replaceable, with the replacement of the controller board 125 able to change EMR wavelength, pulse dosage, treatment duration, and power.

An independent safety test was conducted to assess the device 100 regarding relevant exposure limits and risk group classification. The assessment of the device 100 was conducted under the IEC 62471 and 60601-2-57 standards, showing that the device 100 EMR output falls under the “exempt” risk group classification, well within the allowed exposure limits in terms of eye safety.

### Examples

In Vitro Studies

A series of in vitro studies were conducted to elucidate the specific biochemical interactions that produce a beneficial effect by application of light emitted by the device described above on human inflammatory cells and proteins. The putative mechanism of action is described in more detail below.

The inventors conducted an in vitro study in collaboration with HemaCare, a Charles River company, to measure how the light regimen of the device described above affect the production of proinflammatory substances by cells in the upper airway. Human Peripheral Blood Mononuclear Cells (PBMCs) were stimulated with LPS and various cytokines and histamine were measured before and after stimulation with and without exposure to light as described below. Half of the cell cultures received medium containing lipopolysaccharide (LPS) to stimulate secretion of inflammatory mediators. Triplicate wells of cells were exposed to light one hour after LPS was added. Culture medium were collected after 4 hours and after 48 hours.

Two light exposures were tested: the cultures were exposed to the light emitted by the device described above having at least one wavelength from about 660 nm, about 850 nm, and about 940 nm for 6 seconds, 12 seconds, 18 seconds and 24 seconds, and responses were measured. The nominal distance from the cells was 50 mm and the min-max values received by the cells were min 8.4 mJ/cm² to max 33.6 mJ/cm².

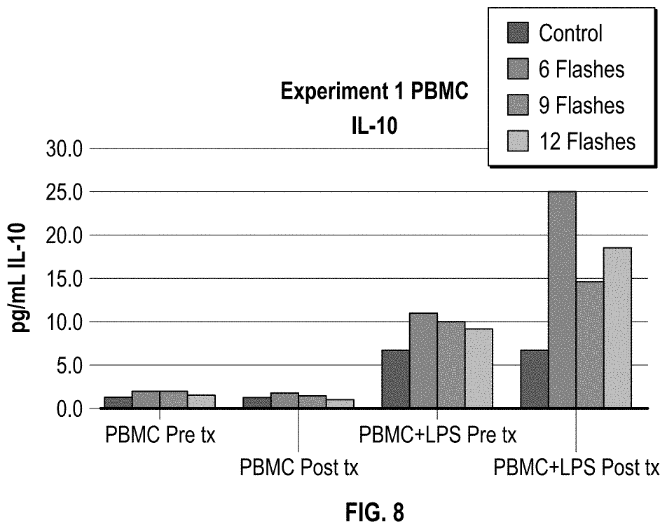

The samples collected after 4 hours were tested for the presence of leukotriene B4, histamine and TNFα. The samples collected after 48 hours were tested for IL-10, IL-12p70, IL-10, and IL-6. The graph of FIG. 8 shows that IL-10 is increased post treatment at various endpoints, and hence is indicative of a mechanism of action defining a therapeutic effect.

Those cells are part of the human immune response during an allergic episode. The cells and proteins play essential roles in the human nasal cavity's immune (over)response when it is exposed to allergens: some rush to the rescue with protective exuberance while others look to keep the response under control and lead nasal functions back to their normal selves.

The results of the preliminary study provide a glimpse of how the human immune system goes to battle to fend off unwanted allergens. Certain proteins, and in particular Interleukin-10 (IL-10), that help to control inflammatory reactions during an allergic fit or episode, responded immediately and favorably to the dosage of light provided by the device described above. The results showed a decrease in histamine loads and an increase in Interleukin-10 (IL-10). This could be significant because histamines is the most prominent protein acting out, and IL-10 is known to serve as a potent down-regulator of inflammatory responses and plays a critical role in controlling allergic airway inflammation.

There is ample data in the literature that support the role of IL-10 as a natural dampener of the immune response in a complex dance of agents that induce, stimulate and others that downregulate and turn off ongoing immune cell activation and inflammation. Additional detail is provided herein.

These findings provide a rationale for how light works to help manage the symptoms of seasonal allergies, and support that light therapy offers an efficient solution earlier in the allergic cycle and even help the nasal cavity prime itself against an invasion of allergens.

Pathophysiological Responses of the Nasal Mucosa in Allergic Rhinitis

Allergic rhinitis is a response of the nasal passages to specific allergens which is mediated by the antibody IgE.

It is a type 1 hypersensitivity reaction which occurs when the nasal mucosa becomes sensitized to a specific environmental allergen. Common environmental allergens which cause rhinitis include mites, pollen, animals and fungi. With future exposure to the antigen, sensitized individuals experience an abnormal and pathological inflammatory response and often also experience a similar reaction to unrelated allergens like tobacco smoke or pollution.

IgE is Produced by B Cells in the Nasal Mucosa.

The IgE antibodies produced inhabit immune system cells called mast cells and basophils which would not normally consider the particles (e.g. dust, pollen) as antigens.

Mast cells, which form part of the immune system and mediate the inflammatory response, are abundant in the nasal mucosa of sensitized (allergic) individuals.

Basophils, which release cytokines as well as other inflammatory factors called histamines, are normally not present in the nasal mucosa; however, they are evident in the nasal mucosa of individuals affected by allergic rhinitis. The more basophils that are found in the nasal mucosa, the greater the severity of allergic symptoms of allergic rhinitis.

The inflammatory response of the nasal mucosa in allergic rhinitis can be separated into early and late phases.

The pathophysiological response which characterizes allergic rhinitis also induces systemic (whole body) circulation of inflammatory factors which may infiltrate tissues at other sites. Inflammatory factors associated with allergic rhinitis are particularly likely to infiltrate the connecting systems, the upper and the lower airways.

Allergic rhinitis is central to many other conditions. Allergic rhinitis often affects individuals who also experience other allergic conditions including asthma, atopic dermatitis and rhinosinusitis. As the mucosa of the nasal cavity and sinuses are continuous, infection or allergy of the nasal mucosa can spread easily to the sinuses.

Seasonal Allergies

Allergic rhinitis can be triggered by allergens generated by different types of pollen which can be divided into three categories: tree pollen, weed pollen, and grass pollen. The National Allergy Bureau pollen data are grouped into 43 pollen categories: 38 for specific genera and families and five other composite categories: “Total Pollen,” “Other Tree Pollen,” “Other Weed Pollen,” “Other Grass Pollen,” and “Unidentified Pollen.” Ragweed, mountain cedar and hay fever allergies fall under specific genres and families categories.

The inventors undertook a randomized, double-blind placebo-controlled study to evaluate the safety and effectiveness of the device described above in temporarily relieving or reducing the symptoms of mountain cedar and/or ragweed-induced allergic rhinitis, and to evaluate the onset of action and duration of effect of device described above.

Results of an earlier pilot study showed a durable treatment response of 33%. The present pivotal study provides a sample of 194 subjects randomized in a 1:1 ratio (97 subjects randomized to each treatment group) providing a two-sided chi-square test an 80% power to detect a difference in durable response rate of 33% in the group treated with the device of the present invention delivering a set dosage of visible and near infrared vs 15% response rate in the group treated with the matching device delivering a set dosage of visible light providing no therapeutic at 5% significance level.

The primary objective of the study is to evaluate the efficacy of the device in subjects with Allergic Rhinitis. Secondary objectives include an evaluation of the safety and tolerability of the device in subjects with Allergic Rhinitis, and to evaluate the onset of action and duration of effect of the device.

Subjects must stop the use of any devices and medications that are used for the treatment of symptoms of Allergic Rhinitis and observe washout requirements prior to entering screening. Subjects will be evaluated during a screening period of 7 days. Assessment of disease activity will be performed by the subjects twice daily, once in the morning and another in the evening, to confirm eligibility using reflective total nasal symptom score (rTNSS-12). Subjects rate each of the following four symptoms on a scale from 0 (no symptoms) to 3 (severe symptoms) during the preceding 12 hours: nasal congestion, runny nose, sneezing, and itchy nose. Subjects record their morning and evening rTNSS-12 in a diary.

Morning and evening rTNSS-12 are averaged to obtain a reflective total nasal symptom score (rTNSS-24) for the day. Subjects with mean rTNSS-24 of 6 or greater during the screening period are randomized in 1:1 ratio to receive treatment with the device delivering a set dosage of visible and near infrared for six seconds per nostril once a day or a matching device delivering a visible light with no therapeutic effect. Randomization is stratified by age (12 to less than 18 years, 18 to less than 50 years vs. 50 years or older) and baseline rTNSS-24 (6 to 8 vs. greater than 8). Subjects who use prohibited medications during the screening period will be considered screening failure and are not randomized.

Each subject is treated once daily for 14 days. As in the screening period, subjects are asked to record their morning and evening rTNSS-12 in a diary. Subjects who use prohibited medications during the treatment period are considered treatment failure. All subjects are followed for 28 days after their last treatment, at which time physical and rhinoscopic examinations will be performed to document the subject's medical condition and assess their nasal mucosa.

Results of this study are expected to provide durable responses. This is indicative of successful therapy for allergic rhinitis, and as a proxy for other medical conditions described herein.

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

What is claimed is:

1. A device for treatment of a medical condition, the device comprising:
a body;
an electromagnetic radiation source within the body that generates electromagnetic radiation of at least two discreet, non-contiguous wavelengths from about 400 nm to about 1000 nm, with the electromagnetic radiation source being at least one of a laser and a light-emitting diode;
an applicator;
optionally at least one skin contact sensor adjacent to the applicator; and
a power source providing power to the electromagnetic radiation source.

2. The device of claim 1, wherein the applicator may be at least one of a delivery nozzle and a wand, with the delivery nozzle optionally taking on a conical shape and the wand optionally taking on a cylindrical shape.

3. The device of claim 1, wherein the electromagnetic radiation wavelengths include at least one wavelength from about 400-700 nm, about 850-870 nm, and about 920-950 nm, and optionally the electromagnetic radiation source emits a broad spectrum white light in addition to the electromagnetic radiation wavelengths.

4. The device of claim 3, wherein the electromagnetic radiation wavelengths are about 660 nm, about 850 nm, and about 940 nm, and optionally the electromagnetic radiation source emits a broad spectrum white light in addition to the electromagnetic radiation wavelengths.

5. The device of claim 1, further comprising an on/off button, a cap, and a controller board, with the controller board comprising at least one of a triggering unit, a capacitor, and electronic timing circuitry.

6. The device of claim 5, wherein the applicator is interchangeable with a second applicator that emits a different electromagnetic radiation wavelength or combination of electromagnetic radiation wavelengths and the electromagnetic radiation source emits the electromagnetic radiation wavelengths based on programming from the controller board, with the electromagnetic radiation wavelengths being selectable.

7. The device of claim 6, wherein the electromagnetic radiation source emits the electromagnetic radiation wavelengths based on programming from the controller board, with the electromagnetic radiation wavelengths being selectable.

8. The device of claim 1, wherein the electromagnetic radiation is emitted as a pulse between about 1 pulse per treatment and about 30 pulses per treatment, the electromagnetic radiation source is placed within 60 mm from a distal end of the applicator, the pulse duration ranges from about 1 millisecond to about 1000 milliseconds, and the frequency of pulses ranges between about 1 Hertz and about 10 Hertz.

9. The device of claim 8, wherein the pulse duration ranges from about 10 milliseconds to about 100 milliseconds.

10. The device of claim 1, wherein the electromagnetic radiation may be applied from about 0.5 mJ/cm² to about 0.5 J/cm².

11. The device of claim 1, wherein the electromagnetic radiation may be applied from about 0.75 mJ/cm² to about 0.25 J/cm².

12. The device of claim 1, wherein the electromagnetic radiation may be applied at about 1.0 mJ/cm².

13. A method of treating a condition, the method comprising:
i. providing a device for treatment of a condition, the device comprising:
a body;
an electromagnetic radiation source within the body that generates electromagnetic radiation of at least one wavelength from about 400 nm to about 1000 nm;
an applicator;
optionally at least one skin contact sensor adjacent to the applicator; and
a power source providing power to the electromagnetic radiation source.
ii. aiming the applicator towards an area of interest;
iii. emitting electromagnetic radiation from the applicator at the at least one wavelength from about 400 nm to about 1000 nm;
wherein the condition may be at least one of allergic rhinitis, non-allergic rhinitis, perennial allergies, seasonal allergies, ragweed allergies, sinusitis, cold sores, acne vulgaris, and herpes, the electromagnetic radiation is filtered so that its wavelength falls within a defined range, and the electromagnetic radiation is non-thermal.

14. The method of claim 13, wherein the electromagnetic radiation is emitted as at least one pulse, with the at least one pulse duration ranging from about 1 millisecond to about 1000 milliseconds.

15. The method of claim 14, wherein the at least one pulse duration ranges from about 10 milliseconds to about 100 milliseconds.

16. The method of claim 13, wherein the electromagnetic radiation may be applied from about 0.5 mJ/cm² to about 0.5 J/cm².

17. The method of claim 13, wherein the electromagnetic radiation may be applied from about 0.75 mJ/cm² to about 0.25 J/cm².

18. The method of claim 13, wherein the electromagnetic radiation may be applied at about 1.0 mJ/cm².

19. A kit for treating a condition, the kit comprising:
i. a device for treatment of a condition, the device comprising:
a body;
an electromagnetic radiation source within the body that generates electromagnetic radiation of at least one wavelength from about 400 nm to about 1000 nm;
an applicator;
optionally at least one skin contact sensor adjacent to the applicator; and
a power source providing power to the electromagnetic radiation source within the body;
ii. instructions that explain how to use the device;
wherein the device further comprises a cap, the condition may be at least one of allergic rhinitis, non-allergic rhinitis, perennial allergies, seasonal allergies, ragweed allergies, hay fever, mountain cedar allergies, sinusitis, cold sores, acne vulgaris, and herpes.

20. A device for treatment of a medical condition of a user, the device comprising:
a body;
a delivery nozzle;
at least one light emitting diode in the delivery nozzle that generates electromagnetic radiation of at least two discreet, non-contiguous wavelengths including from about 400-700 nm, about 850-870 nm, and about 920-950 nm,
wherein the electromagnetic radiation is emitted as a pulse between about 1 pulse per treatment and about 60 pulses per treatment, and
wherein frequency of pulses ranges between about 1 Hertz and about 10 Hertz;
optionally at least one skin contact sensor adjacent to the applicator; and
a power source providing power to the electromagnetic radiation source, wherein the electromagnetic radiation is be applied to a treatment site of the user from about 0.5 mJ/cm² to about 0.5 J/cm².
